(19) United States
(12) Patent Application Publication
(10) Pub. No.: US 2019/0374747 A1
(43) Pub. Date: Dec. 12, 2019

(54) MEDICAL APPLIANCE SECURING DEVICE

(71) Applicant: M.C. Johnson Company, Inc., Fort Myers, FL (US)

(72) Inventors: Matthew LeLievre, Estero, FL (US); Victor Ballo, Cape Coral, FL (US)

(21) Appl. No.: 16/439,428

(22) Filed: Jun. 12, 2019

Related U.S. Application Data
(60) Provisional application No. 62/683,788, filed on Jun. 12, 2018.

(51) Int. Cl. A61M 25/02

(52) U.S. Cl. CPC A61M 25/02; A61M 2025/0266; A61M 2025/0253

(57) ABSTRACT

Medical appliance securing device. In one embodiment, the medical appliance securing device includes an anchor and a retaining tab. The anchor is designed to be secured to a surface and includes a pair of patches. The retaining tab is designed to be wrapped around a medical appliance and may extend from and interconnect the patches. Patches of complementary hook and loop fasteners are spaced apart on one face of the retaining tab, and an adhesive patch is positioned on the same face of the retaining tab between the two patches. In this way, the retaining tab can be wrapped around and adhered to the medical appliance and then secured to itself. A patch of loop fasteners is secured to the other face of the retaining tab, and a patch of complementary hook fasteners is secured to one of the patches. Accordingly, the retaining tab can be secured to the anchor.

### CROSS-REFERENCE TO RELATED APPLICATIONS

The present application claims the benefit under 35 U.S.C. 119(e) of U.S. Provisional Patent Application No. 62/683,788 inventors Matthew LeLievre et al., filed Jun. 12, 2018, the disclosure of which is incorporated herein by reference.

### BACKGROUND OF THE INVENTION

The present invention relates generally to medical appliance securing devices and more particularly to a novel medical appliance securing device.

Various medical devices, or portions thereof, are shaped as generally tubular members. Examples of such devices include, but are not limited to, catheters, thermometers, intravenous needles, and various types of electrical wires. There is often a need to secure such devices to or near a patient's body so that the devices can appropriately function. For example, catheters, which are generally long, tubular, flexible conduits used to transport various types of fluids to and from the body of a patient, are often laterally and/or longitudinally fixed to the body of a patient to ensure proper placement and functioning of the catheter, as well as to offer some degree of comfort and mobility for the patient. In particular, some medical procedures involving the use of catheters require the application of a tensile force, known as traction, to the catheter, thus making it particularly necessary to firmly secure the catheter in its longitudinal direction.

To secure a catheter or other medical appliance in the manner indicated above, practitioners have often used one or more strips of conventional, medical-grade, adhesive tape to secure a length of the medical appliance directly to the patient's skin. However, this method has its shortcomings as the adhesive tape tends to become loose over time. In addition, each time that one wishes to adjust the placement of the medical appliance or each time that one wishes to remove the medical appliance from the patient, the adhesive tape must be removed from the skin of the patient, thereby frequently causing irritation and discomfort to the patient.

Consequently, various types of medical appliance securing devices have been devised in an effort to provide the desired restraint and to overcome the disadvantages associated with the use of strips of adhesive tape. For example, in U.S. Pat. No. 4,165,748, inventor Johnson, which issued Aug. 28, 1979, and which is incorporated herein by reference, there is disclosed a simple, easily applied, catheter securing device. The catheter securing device includes two main parts having adhesive thereon for temporary attachment to the limb of a patient. In addition, the device includes a narrow bridge connecting the two main parts. The narrow bridge is also provided with adhesive and is foldable on itself to form a double member. Fasteners, such as snap fasteners or VELCRO® hook and loop fasteners, are positioned on the narrow bridge in such a way that the catheter tube may be releasably held by the narrow bridge in a desired orientation to a patient.

Another example of a medical appliance securing device is disclosed in U.S. Pat. No. 4,976,700, inventor Tollini, which issued Dec. 11, 1990, and which is incorporated herein by reference. In this patent, there is disclosed a securing device for securing to a patient's skin or to a support, a medical device such as tubing, a catheter, an intravenous needle, or the like, including an elongated tape having base portions and a central tab formed integrally therewith, pressure-sensitive tape on the base portions and on an exposed window of the tab, and hook and pile fastener portions on opposite sides of the exposed adhesive on the tab and on the base portion facing the exposed adhesive. A method of fabricating a securing device consisting of the steps of providing a strip of pressure-sensitive tape with release paper thereon, cutting out a window in a central portion of the strip of tape, removing release paper from the central portion of the pressure-sensitive tape, folding the central portion on itself to cause the facing exposed pressure-sensitive adhesive parts to adhere to each other and to provide a window of pressure-sensitive tape defined by the window which was cut out with the remainder of the strip forming a base, and securing hook and pile fastener material on opposite sides of the window of pressure-sensitive tape on the tab and on the portion of the securing device adjacent thereto which constitutes a base.

Still another example of a medical appliance securing device is disclosed in U.S. Pat. No. 5,147,322, inventors Bowen et al., which issued Sep. 15, 1992, and which is incorporated herein by reference. In this patent, there is disclosed a securing device for laterally and longitudinally securing generally tubular members having various diameters to any desired location on the surface of a patient's skin or other support. The device comprises an anchoring patch having one surface coated with adhesive for bonding the device to a patient's skin or some other support. A retaining tab is connected to the anchoring patch and contains an aperture such that the retaining tab may be wrapped around the circumference of the tubular member, inserted through the aperture, and firmly secured to the anchoring patch through the use of fastening means.

Still yet another example of a medical appliance securing device is disclosed in U.S. Pat. No. 5,304,146, inventors Johnson et al., which issued Apr. 19, 1994, and which is incorporated herein by reference. In this patent, there is disclosed a device for securing a generally tubular member of a medical appliance to a support surface. In one embodiment, the device comprises an anchoring patch, the anchoring patch including a first segment and a second segment and having a top surface and a bottom surface. The bottom surface is coated with an adhesive for attaching the anchoring patch to the support surface. A pair of flexible retaining tabs extend from and interconnect the inner edges of the first and second segment, the flexible retaining tabs being of sufficient length to helically wrap around the circumference of the generally tubular member and contact the top surface of the anchoring patch. First fasteners are secured to the free ends of the flexible retaining tabs and a pair of complementary fasteners are spaced outwardly relative to the tabs and are secured to the top surface of said anchoring patch. A generally tubular member may be retained in the device either by helically wrapping the flexible retaining tabs around the circumference of the generally tubular member and then coupling together the fasteners or by positioning the generally tubular member between the flexible tabs and the complementary fasteners and looping the retaining tabs over the generally tubular member and then coupling together the fasteners.

Still yet another example of a medical appliance securing device is disclosed in U.S. Pat. No. 6,419,660, inventor Russo, which issued Jul. 16, 2002, and which is incorporated herein by reference. In this patent, there is disclosed a tube holder and a method for manufacturing the tube holder. The tube holder includes a base for attachment to a surface, for example, a patient's skin, and a tab for securing the tube to the base. According to one embodiment, the tube holder includes a first layer having first and second sides and first and second sections, and a second layer having first and second sides and first and second sections. The first sides of the first and second layers are attached to one another in the first sections of the first and second layers, the second sections of the first sides of the first and second layers are unattached to one another, and the first sections of the first and second layers form the tab and the second sections form the base. The tube holder also includes a third layer attached to the second side of the first layer for receiving a tube.

Still a further example of a medical appliance securing device is disclosed in U.S. Patent Application Publication No. US 2006/0041233 A1, inventor Bowen, which was published Feb. 23, 2006, and which is incorporated herein by reference. In this patent application, there is disclosed an apparatus for releasably securing an appliance on or adjacent a person. The apparatus includes a base and a flap or tongue attached to the base. The tongue may be wrapped around portions of an appliance and then attached to the base to releasably secure the appliance to the base. The base is formed from a first layer of material. The tongue is formed from a second layer of material disposed on the first layer. One or more of the layers is formed from medical grade adhesive tape or any other type of generally flexible material compatible with placement on a person's skin. Some embodiments may include multiple tongues to releasably secure one appliance or multiple appliances to a single base.

Still yet a further example of a medical appliance securing device is disclosed in U.S. Pat. No. 9,233,228 B1, inventors LeLievre et al., which issued Jan. 12, 2016, and which is incorporated herein by reference. In this patent, according to one embodiment, the device includes a pair of anchoring patches, each of the anchoring patches having an inner edge. A retaining tab extends from and interconnects the inner edges of the anchoring patches. The retaining tab has a first end fixed to the patches and a second end that is free to move relative to the patches so that the retaining tab may be wrapped around at least a portion of a medical appliance. Complementary fasteners are adhered to the fixed and free ends of the retaining tab. Both the anchoring patches and the retaining tab are made up of a multi-layer material that includes a breathable non-woven fabric, a pressure-sensitive adhesive adhered to one surface of the fabric, and a moisture-barrier layer adhered to the opposite surface of the fabric.

An example of a commercially available medical appliance securing device is the CATH-SECURE® medical appliance securing device, which is available from the present applicant/assignee, M.C. Johnson Company, Inc. (Ft. Myers, Fla.). The CATH-SECURE® medical appliance securing device, which corresponds generally to the device of U.S. Pat. No. 4,165,748, includes a sheet of DURAPORE® surgical tape that is cut to define two anchoring patches interconnected by a narrow bridge. (DURAPORE® surgical tape, which is commercially available from the 3M Company (Maplewood, Minn.), is a tape consisting of a woven, polyester cloth backing having an acrylate pressure-sensitive adhesive applied to one surface thereof.) The narrow bridge is folded onto and adhered to itself to form a double member retaining tab. A patch of VELCRO® hook fasteners is adhered to the fixed end of the aforementioned retaining tab, and a patch of VELCRO® loop fasteners is adhered to the free end of the retaining tab, the patch of VELCRO® hook fasteners and the patch of VELCRO® loop fasteners being positioned on the same side of the retaining tab. The patch of VELCRO® hook fasteners and the patch of VELCRO® loop fasteners are appropriately spaced apart from one another so that a medical appliance may be releasably held by wrapping the retaining tab around the medical appliance and securing the patches of VELCRO® hook and loop fasteners to one another. The retaining tab of the CATH-SECURE® medical appliance securing device has a length less than 2.5 inches.

Although the aforementioned CATH-SECURE® medical appliance securing device functions reasonably well at restraining longitudinal movement of a secured medical appliance relative to a patient or stationary surface, the present inventors have observed that the secured medical appliance is still able to move laterally since the retaining tab to which it is secured is also able to move laterally. In many instances, such lateral movement of the medical appliance is undesirable.

### SUMMARY OF THE INVENTION

It is an object of the present invention to provide a novel medical appliance securing device.

It is another object of the present invention to provide a medical appliance securing device that overcomes at least some of the shortcomings associated with existing medical appliance securing devices.

It is still another object of the present invention to provide a medical appliance securing device that is easy to manufacture and easy to use.

Therefore, according to one aspect of the invention, there is provided a medical appliance securing device, the medical appliance securing device comprising (a) an anchor, the anchor being adapted to be secured to a surface; (b) a retaining tab, the retaining tab comprising a first end and a second end, the first end being fixed to the anchor, the second end being free to move relative to the anchor and being appropriately spaced from the first end to permit the retaining tab to be wrapped around at least a portion of a medical appliance; (c) a first fastener, the first fastener being disposed on the retaining tab; (d) a second fastener, the second fastener being disposed on the retaining tab, wherein the first fastener and the second fastener are mateable with one another and are appropriately positioned on the retaining tab to enable the retaining tab to be wrapped around the medical appliance and the first and second fasteners to be mated with one another; (e) a third fastener, the third fastener being disposed on the retaining tab; and (f) a fourth fastener, the fourth fastener being disposed on the anchor, wherein the third and fourth fasteners are mateable with one another and are appropriately positioned to enable the retaining tab, with the medical appliance secured thereto, to be secured to the anchor by mating the third and fourth fasteners.

In a more detailed feature of the invention, the anchor may comprise an adhesive layer.

In a more detailed feature of the invention, the first fastener and the second fastener may be complementary hook and loop fasteners.

In a more detailed feature of the invention, the first fastener and the second fastener may be spaced apart from one another, the first fastener may be proximate to the first end of the retaining tab, and the second fastener may be proximate to the second end of the retaining tab.

In a more detailed feature of the invention, the retaining tab may have two opposing faces, and the first fastener and the second fastener may be disposed on a first one of the two opposing faces.

In a more detailed feature of the invention, the medical appliance securing device may further comprise a fifth fastener, and the fifth fastener may comprise an adhesive patch disposed between the first fastener and the second fastener on the first one of the two opposing faces.

In a more detailed feature of the invention, the third fastener may be disposed on a second one of the two opposing faces of the retaining tab.

In a more detailed feature of the invention, the third fastener and the fourth fastener may be complementary hook and loop fasteners.

According to another aspect of the invention, there is provided a medical appliance securing device, the medical appliance securing device comprising (a) an anchor, the anchor being adapted to be secured to a surface; (b) a retaining tab, the retaining tab comprising a first end, a second end, and two opposing faces, the first end being fixed to the anchor, the second end being free to move relative to the anchor and being appropriately spaced from the first end to permit the retaining tab to be wrapped around at least a portion of a medical appliance; (c) a first set of fasteners, the first set of fasteners being adapted to secure the retaining tab to the medical appliance and comprising at least one fastener disposed on a first of the two opposing faces of the retaining tab; and (d) a second set of fasteners, the second set of fasteners being adapted to secure the retaining tab, with the medical appliance secured thereto, to the anchor and comprising at least one fastener disposed on a second of the two opposing faces of the retaining tab.

In a more detailed feature of the invention, the anchor and the retaining tab may form a unitary structure, and the anchor may comprise an adhesive layer.

In a more detailed feature of the invention, the at least one fastener of the first set of fasteners may comprise a first set of complementary hook and loop fasteners disposed on the first of the two opposing faces of the retaining tab.

In a more detailed feature of the invention, the at least one fastener of the first set of fasteners may further comprise an adhesive patch disposed on the first of the two opposing faces of the retaining tab, and the adhesive patch may be disposed between the first set of complementary hook and loop fasteners.

In a more detailed feature of the invention, the at least one fastener of the first set of fasteners may comprise an adhesive patch.

In a more detailed feature of the invention, the second set of fasteners may further comprise at least one fastener disposed on the anchor, and the at least one fastener disposed on the second of the two opposing faces of the retaining tab and the at least one fastener disposed on the anchor may be mateable with one another.

In a more detailed feature of the invention, the at least one fastener disposed on the second of the two opposing faces of the retaining tab and the at least one fastener disposed on the anchor may be complementary hook and loop fasteners.

According to yet another aspect of the invention, there is provided a method for securing a medical appliance securing device to a surface, the method comprising the steps of (a) providing a medical appliance securing device, the medical appliance securing device comprising (i) an anchor, the anchor being adapted to be secured to a surface, (ii) a retaining tab, the retaining tab comprising a first end, a second end, and two opposing faces, the first end being fixed to the anchor, the second end being free to move relative to the anchor and being appropriately spaced from the first end to permit the retaining tab to be wrapped around at least a portion of a medical appliance, (iii) a first set of fasteners, the first set of fasteners being adapted to secure the retaining tab to the medical appliance and comprising at least one fastener disposed on a first of the two opposing faces of the retaining tab, and (iv) a second set of fasteners, the second set of fasteners being adapted to secure the retaining tab to the anchor and comprising at least one fastener disposed on a second of the two opposing faces of the retaining tab, (b) securing the anchor to the surface; (c) securing the retaining tab to the medical appliance using the first set of fasteners; and (d) securing the retaining tab to the anchor using the second set of fasteners.

In a more detailed feature of the invention, the step of securing the retaining tab to the medical appliance may be performed before the retaining tab is secured to the anchor.

In a more detailed feature of the invention, the at least one fastener of the first set of fasteners may comprise a first set of complementary hook and loop fasteners disposed on the first of the two opposing faces of the retaining tab.

In a more detailed feature of the invention, the second set of fasteners may further comprise at least one fastener disposed on the anchor, and the at least one fastener disposed on the second of the two opposing faces of the retaining tab and the at least one fastener disposed on the anchor may be mateable with one another.

In a more detailed feature of the invention, the at least one fastener disposed on the second of the two opposing faces of the retaining tab and the at least one fastener disposed on the anchor may be complementary hook and loop fasteners.

For purposes of the present specification and claims, various relational terms like “top,” “bottom,” “proximal,” “distal,” “upper,” “lower,” “front,” and “rear” are used to describe the present invention when said invention is positioned in or viewed from a given orientation. It is to be understood that, by altering the orientation of the invention, certain relational terms may need to be adjusted accordingly.

Additional objects, as well as features and advantages, of the present invention will be set forth in part in the description which follows, and in part will be obvious from the description or may be learned by practice of the invention. In the description, reference is made to the accompanying drawings which form a part thereof and in which is shown by way of illustration various embodiments for practicing the invention. The embodiments will be described in sufficient detail to enable those skilled in the art to practice the invention, and it is to be understood that other embodiments may be utilized and that structural changes may be made without departing from the scope of the invention. The following detailed description is, therefore, not to be taken in a limiting sense, and the scope of the present invention is best defined by the appended claims.

### BRIEF DESCRIPTION OF THE DRAWINGS

The accompanying drawings, which are hereby incorporated into and constitute a part of this specification, illustrate various embodiments of the invention and, together with the description, serve to explain the principles of the invention. These drawings are not necessarily drawn to scale, and certain components may have undersized and/or oversized dimensions for purposes of explication. In the drawings wherein like reference numerals represent like parts:

FIG. 1 is a side view of one embodiment of a medical appliance securing device constructed according to the teachings of the present invention;

FIG. 2 is a front view, broken away in part, of the medical appliance securing device of FIG. 1, with the medical appliance securing device being folded so that the two anchoring patches are situated back-to-back to one another and with the two anchoring patches lying substantially coplanar with the retaining tab;

FIG. 3 is a rear view of the medical appliance securing device of FIG. 1, with the medical appliance securing device being folded so that the two anchoring patches are situated back-to-back to one another and with the two anchoring patches lying substantially coplanar with the retaining tab;

FIG. 4 is a fragmentary side view, showing the medical appliance securing device of FIG. 1 secured to a surface and holding a medical appliance but with the retaining tab free to move; and

FIG. 5 is a fragmentary side view, showing the medical appliance securing device of FIG. 1 secured to a surface and holding a medical appliance and with the retaining tab secured to one of the anchoring patches.

### DETAILED DESCRIPTION OF PREFERRED EMBODIMENTS

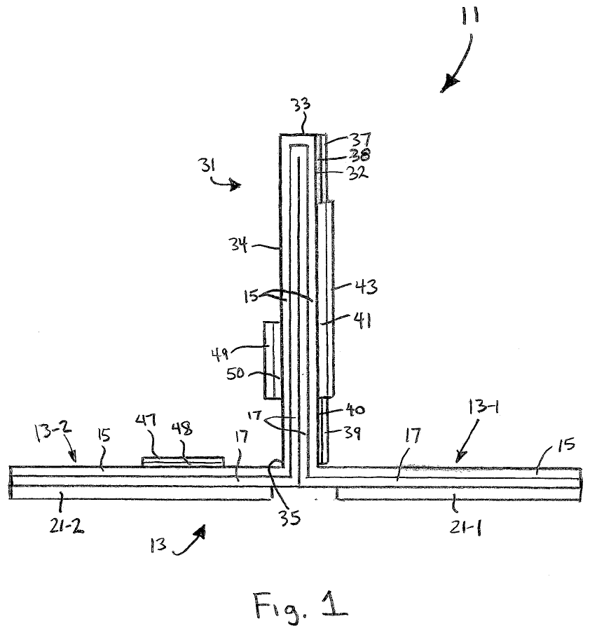

Referring now to FIGS. 1 through 3, there are shown various view of one embodiment of a medical appliance securing device constructed according to the teachings of the present invention, the medical appliance securing device being represented generally by reference numeral 11.

Device 11 may comprise an anchor 13. Anchor 13, in turn, may comprise a pair of patches 13-1 and 13-2. In the present embodiment, each of patches 13-1 and 13-2 is generally rectangular in shape; however, patches 13-1 and 13-2 may assume other shapes, such as those disclosed in U.S. Pat. No. 9,233,228.

Each of patches 13-1 and 13-2 may be a laminate that may comprise a base layer 15 and an adhesive layer 17. Base layer 15 may be a layer of breathable fabric. Although not the case in the present embodiment, base layer 15 may be made of a stretchable material. Adhesive layer 17, which may be applied directly to the bottom surface of base layer 15, may comprise a pressure-sensitive adhesive. An example of a multi-layered material suitable for use in forming patches 13-1 and 13-2 may be DURAPORE® 1538 surgical tape (3M Company, Maplewood, Minn.).

Peelable release liners 21-1 and 21-2 may be removably adhered to the bottoms of patches 13-1 and 13-2, respectively. When one wishes to adhere device 11 to a surface, liners 21-1 and 21-2 may be peeled away, thereby exposing layer 17 of patches 13-1 and 13-2.

Device 11 also may comprise a flexible retaining tab 31. Tab 31 may extend between and may interconnect the inner edges of anchoring patches 13-1 and 13-2. Tab 31 and patches 13-1 and 13-2 preferably constitute a unitary structure, with tab 31 being formed by doubling onto itself and adhering a narrow bridge of material consisting of the same laminate structure used to form patches 13-1 and 13-2. Consequently, tab 31 may comprise a pair of adhesive layers 17 in direct contact with one another, the adhesive layers being sandwiched between a pair of base layers 15. Tab 31 may have a free end 33 and a connected end 35, connected end 35 being connected to the inner edges of patches 13-1 and 13-2. Tab 31 is preferably sufficiently long (e.g., approximately 3.1 inches) to be used in the manner described below.

Device 11 may further comprise one or more fasteners that may be used to secure retaining tab 31 to a medical appliance. In the present embodiment, such one or more fasteners may comprise a patch 37 of VELCRO® hook fasteners secured by an adhesive 38 (or other similar means) to a first face 32 of retaining tab 31 proximate to free end 33, a patch 39 of VELCRO® loop fasteners secured by an adhesive 40 (or other similar means) to face 32 of retaining tab 31 proximate to connected end 35, and a patch 41 of double-sided adhesive tape secured to face 32 of retaining tab 31 and extending between patches 37 and 39. (If desired, the ends of patch 41 may be positioned under patches 37 and 39.) A peelable release liner 43 may be removably adhered over patch 41. Patches 37, 39 and 41 may be designed and retaining tab 31 may be dimensioned so that a medical appliance may be secured to retaining tab 31 by wrapping retaining tab 31 substantially around the circumference of the medical appliance, with patch 41 contacting the medical appliance and with patches 37 and 39 mating with one another.

Although, in the present embodiment, patch 37 may comprise hook fasteners and patch 39 may comprise complementary loop fasteners, it is to be understood that, alternatively, patch 37 may comprise loop fasteners and patch 39 may comprise complementary hook fasteners. Moreover, other types of fasteners, besides complementary hook and loop fasteners, may be used in place of patches 37 and 39, such fasteners including, but not being limited to, snap fasteners, adhesives, including repositionable pressure-sensitive adhesive tapes, and other fastening materials that may permit repeated fastening and unfastening. Furthermore, although, in the present embodiment, each of patches 37, 39 and 41 extends across the entire width of tab 31, one or more of patches 37, 39 and 41 may extend only partially across the width of tab 31. Also, it is to be understood that, in another embodiment, patch 41 may be omitted. (Where layer 15 is made of a stretchable material, tab 31 may stretch in those areas uncovered by a fastener.)

Device 11 may further comprise one or more fasteners used to secure retaining tab 31 to one of patches 13-1 and 13-2. In the present embodiment, such one or more fasteners may comprise a patch 47 of VELCRO® hook fasteners secured by an adhesive 48 (or other similar means) to patch 13-2 and a patch 49 of VELCRO® loop fasteners secured by an adhesive 50 (or other similar means) to face 34 of retaining tab 31 a short distance from connected end 35. Patches 47 and 49 are appropriately situated on patch 13-2 and retaining tab 31, respectively, so that they may be mated, for example, after a medical appliance has been secured to retaining tab 31 in the manner described above.

Although, in the present embodiment, patch 47 may comprise hook fasteners and patch 49 may comprise complementary loop fasteners, it is to be understood that, alternatively, patch 47 may comprise loop fasteners and patch 49 may comprise complementary hook fasteners. In addition, although, in the present embodiment, patch 47 extends across the entire width of patch 13-2 and patch 49 extends across the entire width of tab 31, it is to be understood that patch 47 may extend only partially across the width of patch 13-2 and/or patch 49 may extend only partially across the width of tab 31. Moreover, other types of fasteners, besides complementary hook and loop fasteners, may be used in place of patches 47 and 49, such fasteners including, but not being limited to, snap fasteners, adhesives, including repositionable pressure-sensitive adhesive tapes, and other fastening materials that may permit repeated fastening and unfastening.

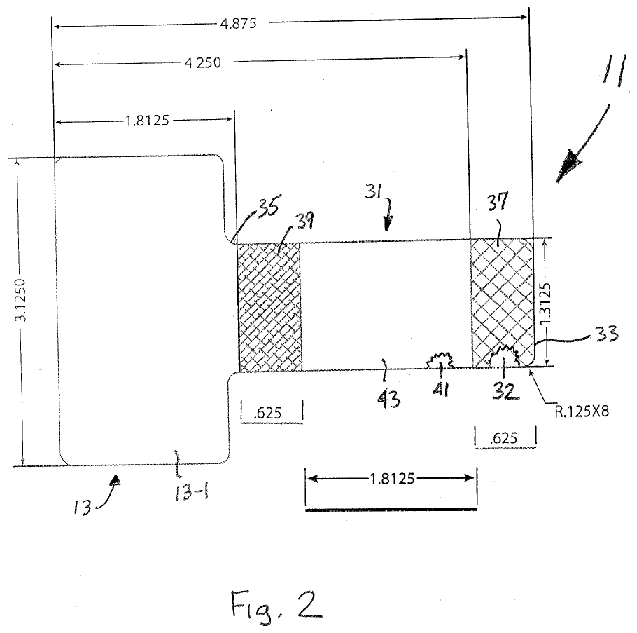

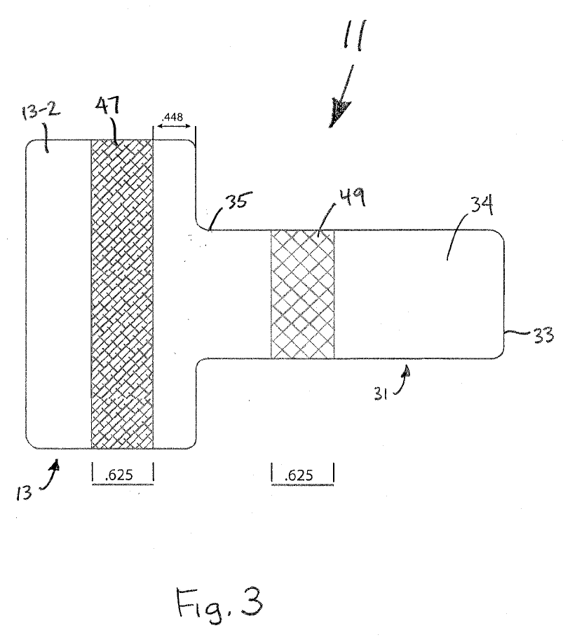

Illustrative dimensions (in inches) for device 11 are shown in FIGS. 2 and 3.

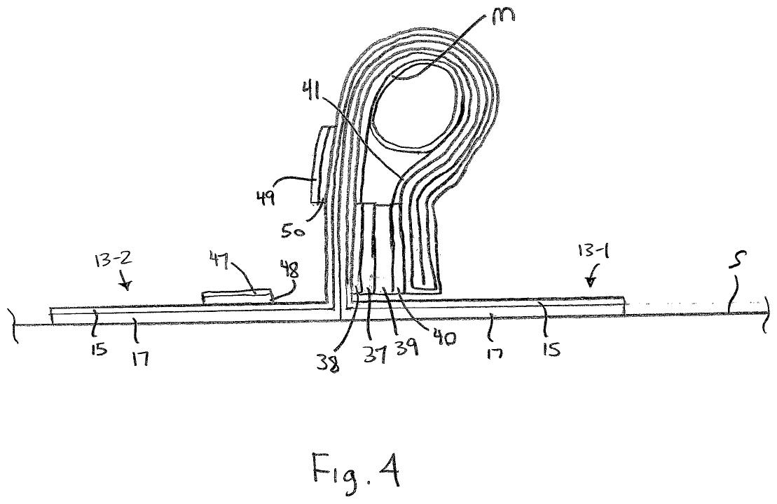

In use, release liners 21-1 and 21-2 may be peeled away from patches 13-1 and 13-2, respectively, thereby exposing adhesive layer 17 of patches 13-1 and 13-2. Next, as seen in FIG. 4, patches 13-1 and 13-2 may be secured to a support surface S, such as a patient's skin or a stationary object (like a bed rail), by pressing adhesive layer 17 against the support surface S until adhesive layer 17 securely bonds thereto. Release liner 43 may then be peeled away from patch 41, a medical appliance M may be secured to patch 41, retaining tab 31 may be tightly wrapped around at least a portion of the circumference of medical appliance M, and fasteners 37 and 39 may be secured to one another. In this manner, medical appliance M may be secured to the stationary object in such a way as to limit longitudinal movement of medical appliance M relative to the stationary object. (It is to be understood that, if desired, retaining tab 31 may be wrapped around medical appliance M and secured to itself with fasteners 37 and 39 without removing release liner 43 and adhering patch 41 to medical appliance M.)

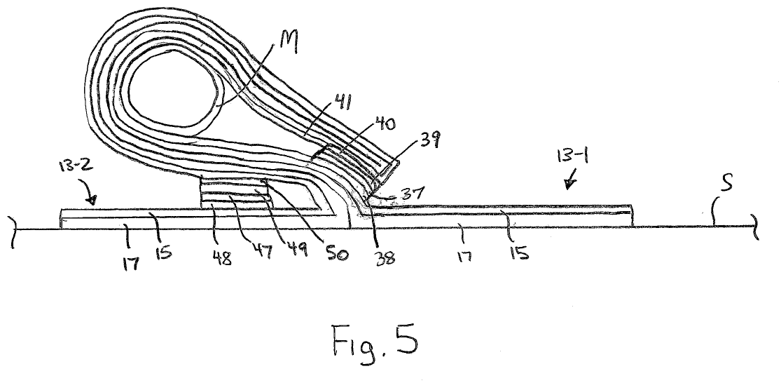

Next, as can be seen in FIG. 5, retaining tab 31 may be secured to patch 13-2 by mating patches 47 and 49. As can be appreciated, the mating of patches 47 and 49 not only constrains the lateral movement of retaining tab 31 but also constrains the lateral movement of medical appliance M, which is secured to retaining tab 31.

Therefore, device 11 is superior to comparable conventional medical appliance securing devices in that device 11 can constrain both longitudinal movement and lateral movement of a medical appliance relative to the stationary object to which it is secured.

It is to be understood that, although the above-described embodiments of the present invention have included a single retaining tab, the present invention is not limited to embodiments having a single retaining tab, but rather, encompasses embodiments having a plurality of retaining tabs, such as in U.S. Pat. No. 5,304,146. Moreover, although the above-described embodiments of the present invention have included a retaining tab of rectangular shape, the present invention is not limited to retaining tabs having a rectangular shape, but rather, encompasses embodiments having a non-rectangular shape, such as in U.S. Pat. No. 5,304,146. Furthermore, the present invention is not limited to the placement of complementary fastening means in the positions shown in the above embodiments, but rather, encompasses at least the various locations disclosed in U.S. Pat. No. 5,304,146.

The embodiments of the present invention described above are intended to be merely exemplary and those skilled in the art shall be able to make numerous variations and modifications to it without departing from the spirit of the present invention. All such variations and modifications are intended to be within the scope of the present invention as defined in the appended claims. cm What is claimed is:

1. A medical appliance securing device, the medical appliance securing device comprising:
(a) an anchor, the anchor being adapted to be secured to a surface;
(b) a retaining tab, the retaining tab comprising a first end and a second end, the first end being fixed to the anchor, the second end being free to move relative to the anchor and being appropriately spaced from the first end to permit the retaining tab to be wrapped around at least a portion of a medical appliance;
(c) a first fastener, the first fastener being disposed on the retaining tab;
(d) a second fastener, the second fastener being disposed on the retaining tab, wherein the first fastener and the second fastener are mateable with one another and are appropriately positioned on the retaining tab to enable the retaining tab to be wrapped around the medical appliance and the first and second fasteners to be mated with one another;
(e) a third fastener, the third fastener being disposed on the retaining tab; and
(f) a fourth fastener, the fourth fastener being disposed on the anchor, wherein the third and fourth fasteners are mateable with one another and are appropriately positioned to enable the retaining tab, with the medical appliance secured thereto, to be secured to the anchor by mating the third and fourth fasteners.

2. The medical appliance securing device as claimed in claim 1 wherein the anchor comprises an adhesive layer.

3. The medical appliance securing device as claimed in claim 1 wherein the first fastener and the second fastener are complementary hook and loop fasteners.

4. The medical appliance securing device as claimed in claim 3 wherein the first fastener and the second fastener are spaced apart from one another, the first fastener being proximate to the first end of the retaining tab, the second fastener being proximate to the second end of the retaining tab.

5. The medical appliance securing device as claimed in claim 1 wherein the retaining tab has two opposing faces and wherein the first fastener and the second fastener are disposed on a first one of the two opposing faces.

6. The medical appliance securing device as claimed in claim 5 further comprising a fifth fastener, the fifth fastener comprising an adhesive patch disposed between the first fastener and the second fastener on the first one of the two opposing faces.

7. The medical appliance securing device as claimed in claim 5 wherein the third fastener is disposed on a second one of the two opposing faces of the retaining tab.

8. The medical appliance securing device as claimed in claim 7 wherein the third fastener and the fourth fastener are complementary hook and loop fasteners.

9. A medical appliance securing device, the medical appliance securing device comprising:
(a) an anchor, the anchor being adapted to be secured to a surface;
(b) a retaining tab, the retaining tab comprising a first end, a second end, and two opposing faces, the first end being fixed to the anchor, the second end being free to move relative to the anchor and being appropriately spaced from the first end to permit the retaining tab to be wrapped around at least a portion of a medical appliance;
(c) a first set of fasteners, the first set of fasteners being adapted to secure the retaining tab to the medical appliance and comprising at least one fastener disposed on a first of the two opposing faces of the retaining tab; and
(d) a second set of fasteners, the second set of fasteners being adapted to secure the retaining tab, with the medical appliance secured thereto, to the anchor and comprising at least one fastener disposed on a second of the two opposing faces of the retaining tab.

10. The medical appliance securing device as claimed in claim 9 wherein the anchor and the retaining tab form a unitary structure and wherein the anchor comprises an adhesive layer.

11. The medical appliance securing device as claimed in claim 9 wherein the at least one fastener of the first set of fasteners comprises a first set of complementary hook and loop fasteners disposed on the first of the two opposing faces of the retaining tab.

12. The medical appliance securing device as claimed in claim 11 wherein the at least one fastener of the first set of fasteners further comprises an adhesive patch disposed on the first of the two opposing faces of the retaining tab, the adhesive patch being disposed between the first set of complementary hook and loop fasteners.

13. The medical appliance securing device as claimed in claim 9 wherein the at least one fastener of the first set of fasteners comprises an adhesive patch.

14. The medical appliance securing device as claimed in claim 9 wherein the second set of fasteners further comprises at least one fastener disposed on the anchor and wherein the at least one fastener disposed on the second of the two opposing faces of the retaining tab and the at least one fastener disposed on the anchor are mateable with one another.

15. The medical appliance securing device as claimed in claim 14 wherein the at least one fastener disposed on the second of the two opposing faces of the retaining tab and the at least one fastener disposed on the anchor are complementary hook and loop fasteners.

16. A method for securing a medical appliance securing device to a surface, the method comprising the steps of:
(a) providing a medical appliance securing device, the medical appliance securing device comprising
(i) an anchor, the anchor being adapted to be secured to a surface,
(ii) a retaining tab, the retaining tab comprising a first end, a second end, and two opposing faces, the first end being fixed to the anchor, the second end being free to move relative to the anchor and being appropriately spaced from the first end to permit the retaining tab to be wrapped around at least a portion of a medical appliance,
(iii) a first set of fasteners, the first set of fasteners being adapted to secure the retaining tab to the medical appliance and comprising at least one fastener disposed on a first of the two opposing faces of the retaining tab, and
(iv) a second set of fasteners, the second set of fasteners being adapted to secure the retaining tab to the anchor and comprising at least one fastener disposed on a second of the two opposing faces of the retaining tab,
(b) securing the anchor to the surface;
(c) securing the retaining tab to the medical appliance using the first set of fasteners; and
(d) securing the retaining tab to the anchor using the second set of fasteners.

17. The method as claimed in claim 16 wherein the step of securing the retaining tab to the medical appliance is performed before the retaining tab is secured to the anchor.

18. The method as claimed in claim 16 wherein the at least one fastener of the first set of fasteners comprises a first set of complementary hook and loop fasteners disposed on the first of the two opposing faces of the retaining tab.

19. The medical appliance securing device as claimed in claim 16 wherein the second set of fasteners further comprises at least one fastener disposed on the anchor and wherein the at least one fastener disposed on the second of the two opposing faces of the retaining tab and the at least one fastener disposed on the anchor are mateable with one another.

20. The medical appliance securing device as claimed in claim 19 wherein the at least one fastener disposed on the second of the two opposing faces of the retaining tab and the at least one fastener disposed on the anchor are complementary hook and loop fasteners.
